(19) United States
(12) Patent Application Publication
(10) Pub. No.: US 2014/0107580 A1
(43) Pub. Date: Apr. 17, 2014

(54) FLUID INJECTING APPARATUS

(71) Applicant: SEIKO EPSON CORPORATION, Tokyo (JP)

(72) Inventor: Yoshihiko MOMOSE, Shiojiri-shi (JP)

(73) Assignee: SEIKO EPSON CORPORATION, Tokyo (JP)

(21) Appl. No.: 14/051,898

(22) Filed: Oct. 11, 2013

(30) Foreign Application Priority Data
Oct. 15, 2012 (JP) 2012-228262

(51) Int. Cl. A61M 5/142

(52) U.S. Cl. CPC A61M 5/14248
USPC 604/151

(57) ABSTRACT

A fluid injecting apparatus includes: a storage portion which stores a fluid to be injected into a living body; and a pump portion which injects the fluid into the living body. The storage portion is provided closer to the living body side than the pump portion. In addition, it is preferable that the pump portion include a tube in which the fluid flows, a plurality of fingers which sequentially press the tube, a cam portion which sequentially moves the fingers, and a motor portion which drives the cam portion to rotate.

### BACKGROUND

1. Technical Field

The present invention relates to a fluid injecting apparatus which injects a fluid.

2. Related Art

An insulin pump which injects insulin into a living body is used in practice. A fluid injecting apparatus such as the insulin pump is fixed to a living body such as a human body and regularly injects a fluid to the living body such as a human body according to a program set in advance.

In JP-A-2010-48121, a micropump which is provided with a transport mechanism including a cam, a finger, and a tube and a reservoir is illustrated, and a structure in which the transport mechanism and the reservoir are adjacent in a plan view is disclosed (FIG. 5).

A fluid injecting apparatus includes a storage portion which is to store a fluid to be injected into a living body. As described above, although the fluid injecting apparatus is fixed to the living body, when the living body moves around, there is concern that the fluid injecting apparatus may come into contact with external objects. In addition, there is concern that the storage portion may be broken.

When the storage portion is broken, the fluid leaks out. When the amount of the fluid is reduced due to the leakage of the fluid at this time, the fluid cannot be injected into the living body. Therefore, it is desirable that the storage portion which stores the fluid be less likely to be broken.

### SUMMARY

An advantage of some aspects of the invention is that a storage portion which stores a fluid is less likely to be broken.

An aspect of the invention is directed to a fluid injecting apparatus including: a storage portion which stores a fluid to be injected into a living body; and a pump portion which injects the fluid into the living body. The storage portion is provided closer to the living body side than the pump portion.

Other features of the invention are clarified by the specification and the attached drawings.

### BRIEF DESCRIPTION OF THE DRAWINGS

The invention will be described with reference to the accompanying drawings, wherein like numbers reference like elements.

FIG. 1 is a perspective view of the entirety of a micropump.

FIG. 2 is an exploded view of the micropump.

FIG. 3 is a perspective plan view of the micropump.

FIG. 4 is a cross-sectional view of the micropump.

FIG. 5 is a perspective view of the inside of a main body.

FIG. 6 is a perspective view of the rear surface of the main body.

FIG. 7 is an exploded perspective view of a cartridge.

FIG. 8 is a perspective view of the rear surface of a cartridge base.

FIG. 9 is a perspective view of the rear surface of the micropump.

FIG. 10 is a diagram illustrating a rotary finger pump.

### DESCRIPTION OF EXEMPLARY EMBODIMENTS

The following is clarified by the description of the specification and the attached drawings.

A fluid injecting apparatus includes: a storage portion which stores a fluid to be injected into a living body; and a pump portion which injects the fluid into the living body. The storage portion is provided closer to the living body side than the pump portion.

Accordingly, since the pump portion is disposed on the outer side than the storage portion with respect to the living body, the storage portion that stores the fluid can be protected by the pump portion. In addition, the storage portion is less likely to be broken.

In the fluid injecting apparatus, it is preferable that the pump portion include a tube in which the fluid flows, a plurality of fingers which sequentially press the tube, a cam portion which sequentially moves the fingers, and a motor portion which drives the cam portion to rotate.

Accordingly, since the tube is sequentially pressed and closed by the fingers, the fluid in the tube can be allowed to flow in a predetermined direction.

In addition, it is preferable that a main body portion and a cartridge portion which can be assembled in one body be provided, the tube and the plurality of fingers be stored in the cartridge portion together with the storage portion, the motor portion and the cam portion be stored in the main body portion, and a cam surface of the cam portion be disposed at a position that opposes a finger end of the finger when the cartridge portion and the main body portion are assembled in one body.

As such, since the fingers and the tube are provided in the cartridge portion, even in a case where a tube having a diameter different from that of the tube is employed, the cartridge portion in which the fingers having a length that matches the diameter of the tube are assembled can be provided. Accordingly, even when the size of the cam is a standardized size, the cam surface of the cam can be appropriately disposed at a position that abuts on the finger end of the finger.

In addition, it is preferable that the storage portion be configured to have an upper portion made of a plastic and a lower portion made of a film, and the upper portion be configured as a curved surface in the cartridge portion.

Accordingly, the film of the storage portion is deformed according to the residual amount of the fluid, and thus the fluid can be squeezed out so as not to be left.

In addition, it is preferable that the lower portion made of the film be processed to have a shape along the curved surface of the upper portion.

Accordingly, even when the amount of fluid in the storage portion is reduced, the lower portion made of the film is deformed along the curved surface of the upper portion, and thus the fluid can be squeezed out so as not to be left.

In addition, it is preferable that the cartridge portion include a first septum for injecting the fluid between the upper portion made of the plastic and the lower portion made of the film in the storage portion.

Accordingly, the fluid can be injected between the upper portion made of the plastic and the lower portion made of the film via the first septum.

In addition, it is preferable that an injecting portion which injects the fluid sent from the cartridge portion into the living body be provided, and the injecting portion include a second septum to which the fluid from the cartridge portion is injected, and a catheter which sends the fluid injected via the second septum to the living body.

Accordingly, the fluid in the cartridge portion can be injected into the living body via the injecting portion.

In addition, it is preferable that the cartridge portion include a needle member that is inserted through the second septum to send the fluid to the injecting portion, and a tip end position of the needle member have the same height as that of the storage portion in a height direction in which the pump portion and the storage portion overlap.

Accordingly, the height difference between the tip end position of the needle member and the position of the storage portion is small, and thus the fluid stored in the storage portion can be transported to the injecting portion via the needle member with little energy.

### Embodiment

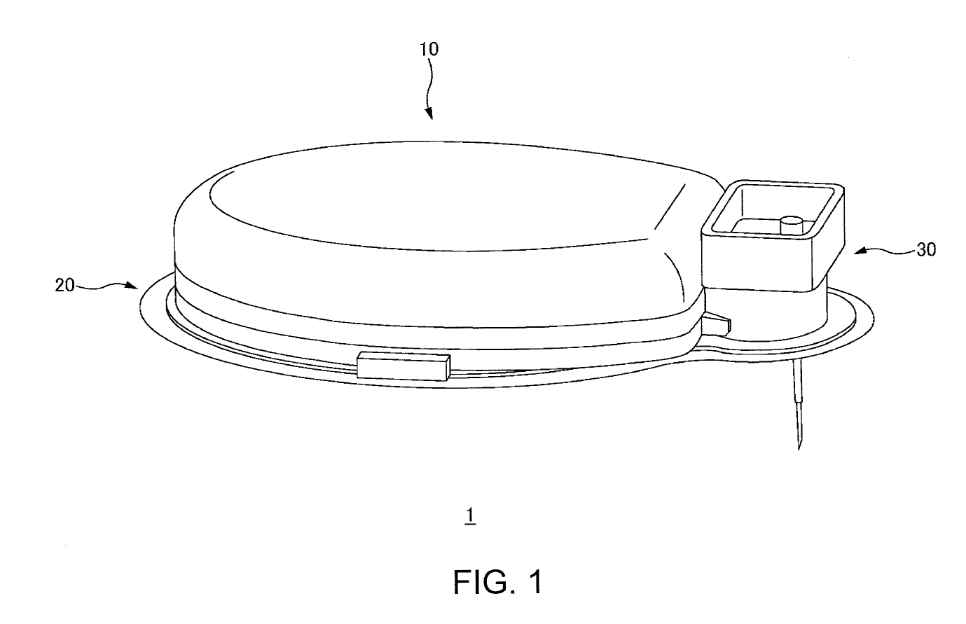

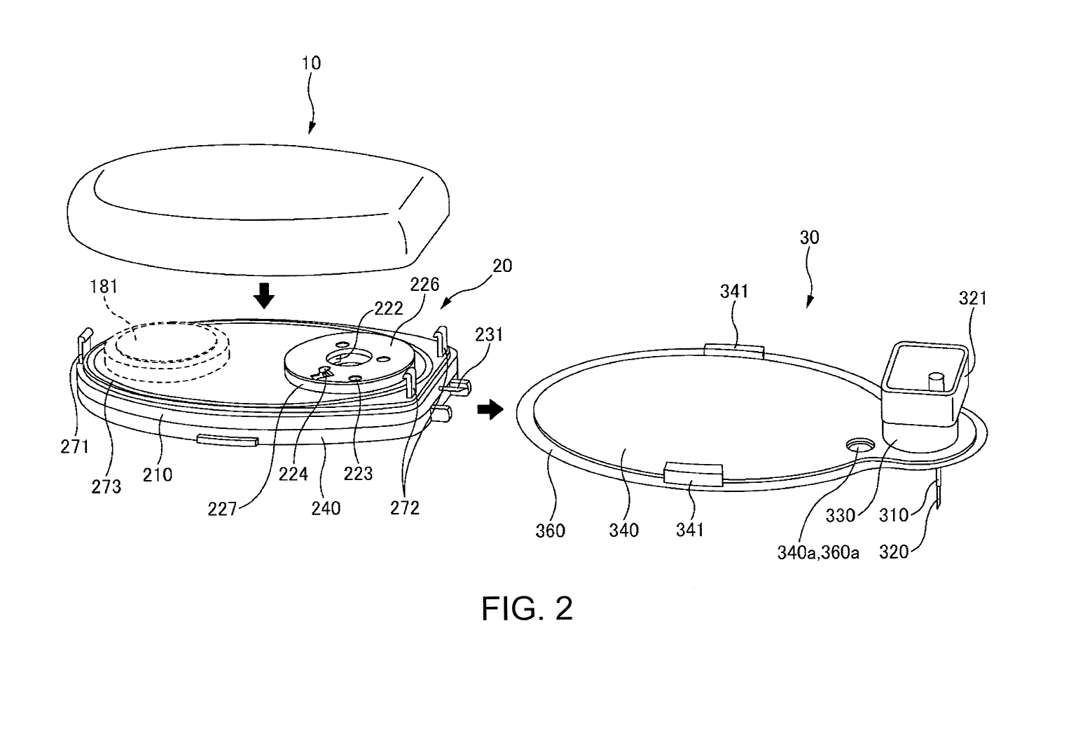

FIG. 1 is a perspective view of the entirety of a micropump 1. FIG. 2 is an exploded view of the micropump 1. The micropump 1 includes a main body 10, a cartridge 20, and a patch 30. The three components can be disassembled as illustrated in FIG. 2 and can be assembled in one body in use as illustrated in FIG. 1. The micropump 1 in this embodiment sticks to a living body and is appropriately used for a regular injection of insulin.

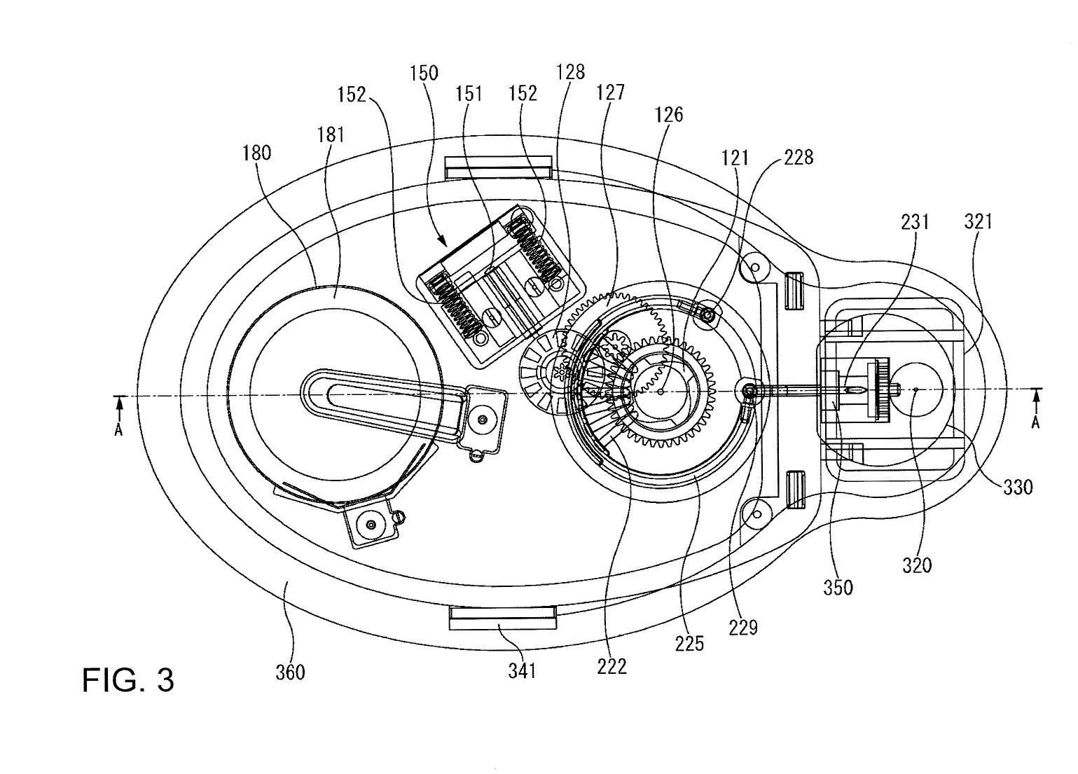

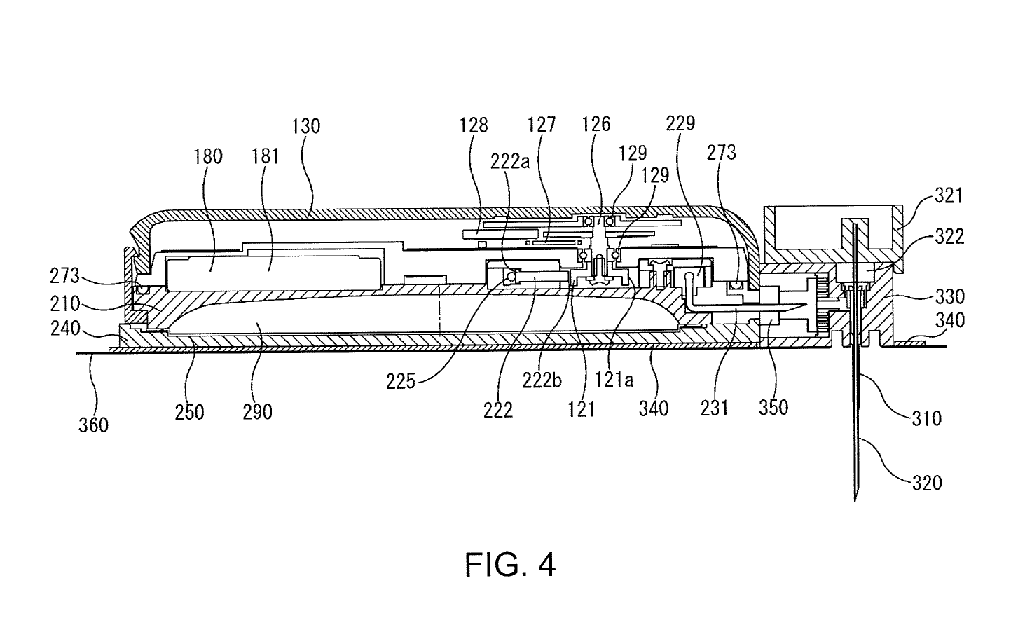

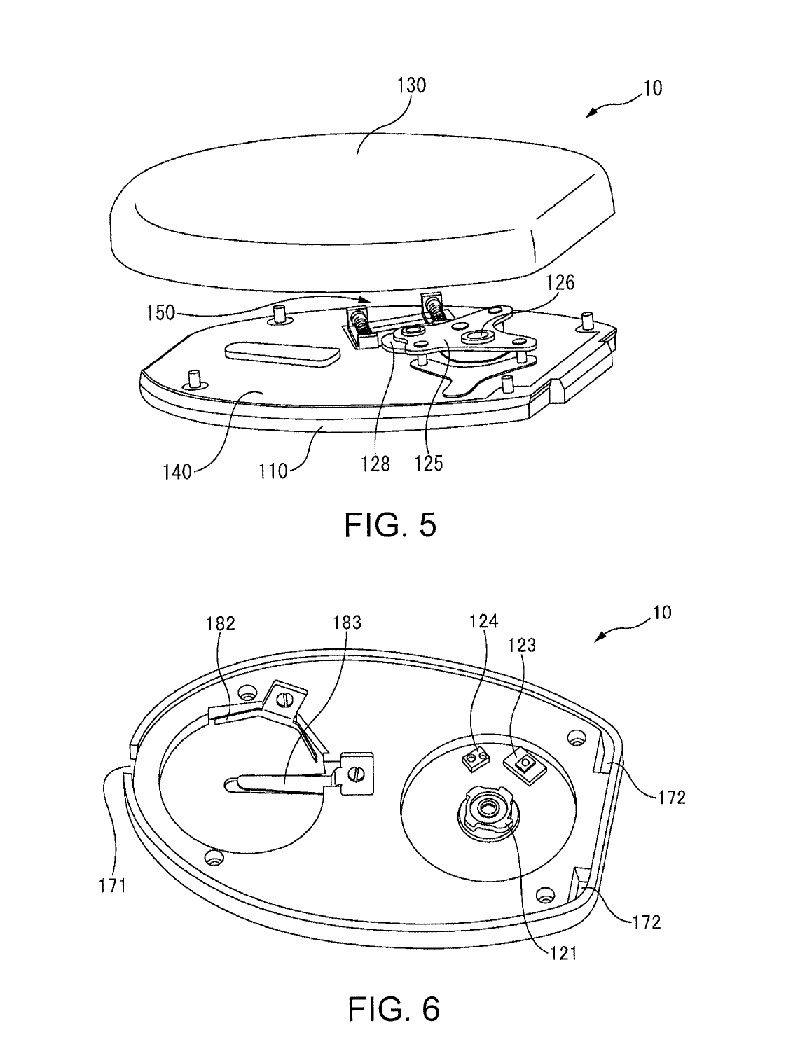

FIG. 3 is a perspective plan view of the micropump 1. FIG. 4 is a cross-sectional view of the micropump 1. That is, FIGS. 3 and 4 are diagrams of the main body 10, the cartridge 20, and the patch 30 which are assembled. FIG. 5 is a perspective view of the inside of the main body 10. FIG. 6 is a perspective view of the rear surface of the main body 10.

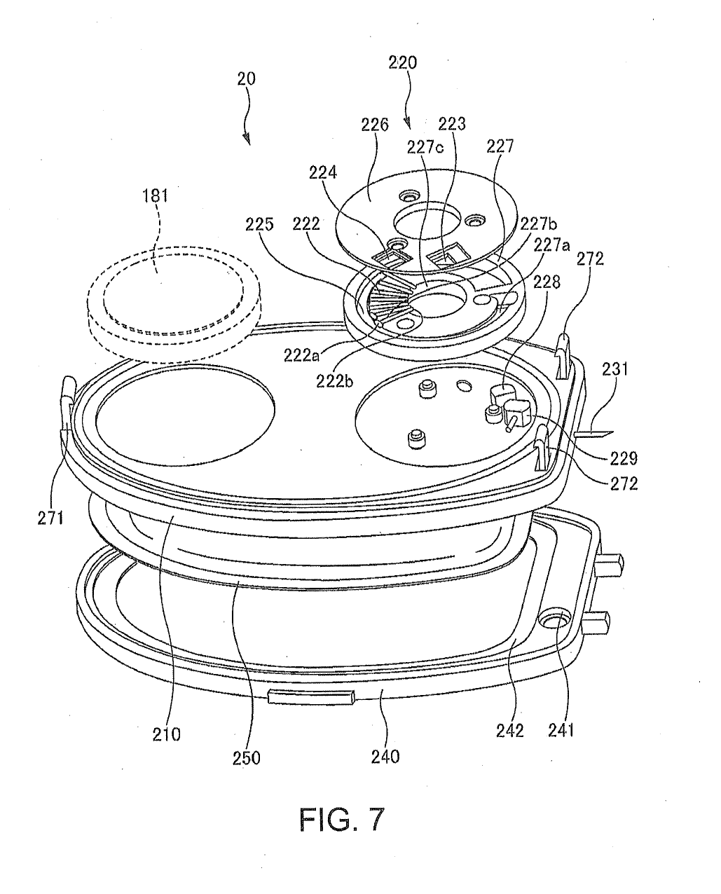

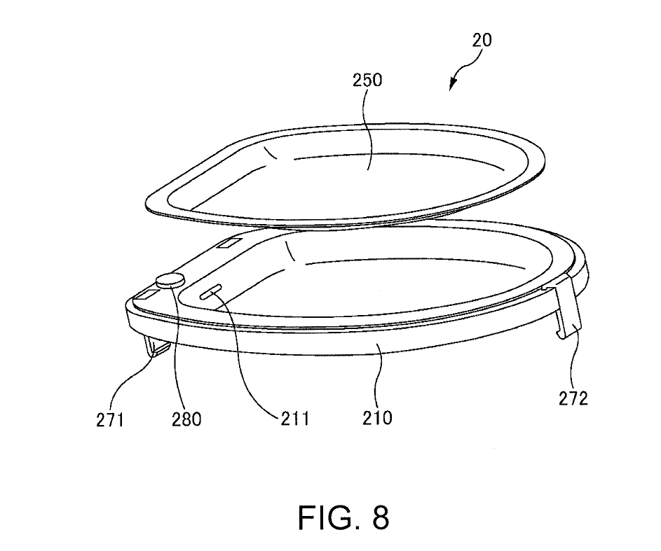

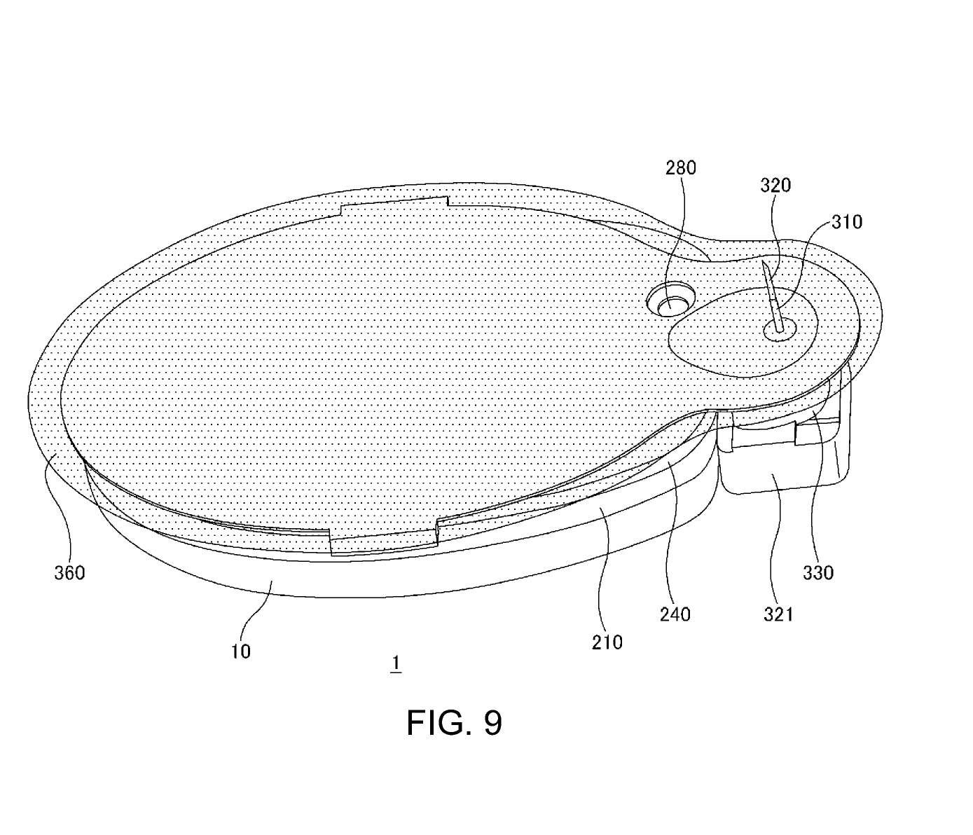

FIG. 6 is a diagram illustrating the rear surface of FIG. 5 described above. FIG. 7 is an exploded perspective view of the cartridge 20. FIG. 8 is a perspective view of the rear surface of a cartridge base 210. FIG. 9 is a perspective view of the rear surface of the micropump 1.

Hereinafter, each part of the micropump 1 will be described with reference to FIGS. 1 to 9 described above. First, each part in the main body 10 (corresponding to a main body portion) will be described.

The main body 10 includes a main body base 110, each part configured on a main body base 110, and a main body case 130. In addition, each part on the main body base 110 is covered by the main body case 130 to be protected.

The main body 10 includes a circuit board 140 configured on the main body base 110. The circuit board 140 is an electronic board for controlling a piezoelectric motor 150 and the like according to programs or the like. In addition, the main body 10 includes the piezoelectric motor 150. The piezoelectric motor 150 is a motor for applying a rotational driving force to a cam 121, which will be described later.

The piezoelectric motor 150 includes a plate-like member 151 and a pair of springs 152 (FIG. 3). The springs 152 impels the plate-like member 151 toward a rotor wheel 128 using their elastic forces. The plate-like member 151 is impelled toward the rotor wheel 128 as described above such that the tip end portion thereof comes into contact with the circumferential surface of the rotor wheel 128.

The plate-like member 151 is a member configured in layers. The plate-like member 151 includes a piezoelectric layer and two electrodes, and the shape thereof is changed by a change in a voltage applied to the two electrodes. For example, longitudinal vibration and flexural vibration are alternately repeated by the applied voltage. Longitudinal vibration changes the length of the plate-like member 151 in the axial direction thereof, and flexural vibration changes the plate-like member 151 in a substantially S shape. As the vibrations are alternately repeated, the rotor wheel 128 is rotated in a predetermined direction.

The rotor wheel 128 has a pinion that is rotated integrally at a position different in the height direction of the micropump 1, and the pinion is engaged with a gear of an intermediate wheel 127 to rotate the intermediate wheel 127. In addition, the intermediate wheel 127 also has a pinion that is rotated integrally at a position different in the height direction of the micropump 1, and the pinion is engaged with a gear that is rotated integrally with an output shaft 126. The rotor wheel 128, the intermediate wheel 127, and the output shaft 126 are fixed to a gear train support 125 fixed to the main body 10 so that each of the shafts thereof can be rotated.

The cam 121 is also fixed to the output shaft 126 pivotally supported by bearings 129 so as to be integrally rotated. In addition, the cam 121 is also rotated along with the rotation of the output shaft 126. Accordingly, the power from the piezoelectric motor 150 is transmitted to the cam 121.

As illustrated in FIG. 6, a hook holder 171 is provided at the front of the main body 10, and hook insertion openings 172 are provided at two points at the rear thereof. A fixing hook 271 of the cartridge 20 is hooked to the hook holder 171, and fixing hooks 272 are hooked to the hook insertion openings 172 so that the cartridge 20 can be fixed to the main body 10 (FIGS. 2 and 4).

At this time, a packing 273 is fitted to a groove portion of the outer periphery of the upper surface of the cartridge base 210. Therefore, when the main body 10 and the cartridge 20 are fixed to each other, a space formed by the main body 10 and the cartridge 20 can be sealed so as not to allow a liquid or the like to infiltrate into the space.

The main body 10 includes a clogging detection element 123 and a bubble detection element 124 at the rear surface thereof (FIG. 6). The clogging detection element 123 includes, for example, a pressure sensor. In addition, when the main body 10 and the cartridge 20 are assembled in one body, the pressure sensor comes into contact with a portion of a tube 225. When the tube 225 is clogged at the downstream side or a position therebelow, the internal pressure of the tube 225 is increased, and the tube 225 itself expands. Therefore, at this time, the tube 225 presses the pressure sensor. Accordingly, by monitoring the pressure detected by the pressure sensor, whether or not the tube is clogged at the downstream side or position therebelow can be determined.

In addition, the bubble detection element 124 includes, for example, an optical sensor. The optical sensor illuminates the tube 225 with light, and detects the reflected light. In addition, the optical sensor can detect a difference between reflected light when a liquid occupies the inside of the tube 225 and reflected light when bubbles occur. Accordingly, whether or not bubbles occur in the tube 225 can be determined.

In addition, the main body 10 includes a secondary battery storage portion 180 at the rear surface thereof (FIG. 6). The secondary battery storage portion 180 has a battery positive terminal 182 and a battery negative terminal 183, and by inserting a secondary battery 181 into the secondary battery storage portion 180, predetermined power can be supplied to each part of the main body 10.

Next, the cartridge 20 (corresponding to a cartridge portion) will be described.

The cartridge 20 includes the cartridge base 210, a cartridge base presser 240, and each part configured on the cartridge base 210. The cartridge base 210 configures a storage portion 290 together with a reservoir film 250 as described later.

The cartridge base 210 of the cartridge 20 includes a finger unit 220 on the upper surface thereof. The finger unit 220 includes a finger base 227, fingers 222, the tube 225, and a finger presser 226. In addition, on the upper surface of the cartridge base 210, a suction connector 228 and a discharge connector 229 are provided. The suction connector 228 is a connector 228 for suctioning a liquid in the finger unit 220, and the discharge connector 229 is a connector for discharging the liquid from the finger unit 220.

A plurality of grooves are formed in the finger base 227, and the suction connector 228 and the discharge connector 229 are inserted into the grooves. In addition, in the finger base 227, a tube guide groove 227a that guides the tube 225 is formed in an arc shape to accommodate the tube 225. One end of the tube 225 is densely connected to the suction connector 228, and the other end thereof is densely connected to the discharge connector 229.

A plurality of finger guides 227b are formed on the inside of the arc formed by the tube guide groove 227a. The finger guides 227b respectively accommodate the fingers 222. Accordingly, a tip end 222a of the finger 222 is disposed to be in a direction substantially perpendicular to the tube 225.

The finger presser 226 is fixed to the upper surface of the finger base 227 by a fixing screw (not illustrated). Accordingly, the finger 222 is able to slide only in a direction along the finger guide 227b.

As described above, since the fingers 222 and the tube 225 are provided on the cartridge 20 side, even in a case where a tube having a diameter different from that of the tube 225 is employed, the cartridge 20 in which the fingers 222 having a length that matches the diameter of the tube are assembled can be provided. Accordingly, even when the size of the cam 121 is a standardized size, a cam surface 121a of the cam 121 can be appropriately disposed at a position that abuts on a rear end portion 222b of the finger 222.

A blogging detection window 223 and a bubble detection window 224 are provided in the finger presser 226. When the main body 10 and the cartridge 20 are assembled, the clogging detection element 123 detects clogging of the liquid in the tube 225 via the clogging detection window 223. In addition, the bubble detection element 124 detects presence or absence of bubbles in the tube 225 via the bubble detection window 224.

A patch connection needle 231 (corresponding to a needle member) is provided at the side surface of the cartridge base 210 to enable the liquid to be sent to the patch 30 via a patch septum 350. The patch connection needle 231 communicates with the discharge connector 229. On the other hand, the suction connector 228 communicates with the storage portion 290, which will be described later, via a through-hole provided in the cartridge base 210. Accordingly, the liquid in the storage portion 290 can be supplied to the patch connection needle 231 through the suction connector 228, the tube 225, and the discharge connector 229.

As illustrated in FIG. 4, in this embodiment, the tip end position of the patch connection needle 231 has substantially the same height as the storage portion 290 in the height direction. Accordingly, although the liquid passes through the tube 225 and the like on the upper surface of the cartridge 20, the height difference itself between the tip end position of the patch connection needle 231 and the position of the storage portion 290 is small. Therefore, the positional energy difference can be reduced, and thus the liquid stored in the storage portion 290 can be sent to the patch connection needle 231 with little energy. This configuration is advantageous in a case where the power saving type piezoelectric motor 150 described above is used.

The cartridge 20 includes the reservoir film 250. The periphery of the reservoir film 250 is pinched between the cartridge base 210 and a film pressing portion 242 provided in the cartridge base presser 240. Accordingly, the storage portion 290 is configured between the reservoir film 250 and the cartridge base 210 such that the liquid can be stored in the storage portion 290.

In addition, the reservoir film 250 may be fixed to the cartridge base 210 by welding to fix the cartridge base presser 240 and the cartridge base 210 to each other.

The cartridge base 210 is made of a plastic and the surface thereof on a side where the reservoir film 250 is provided has a curved surface shape. As such, the storage portion 290 has a curved surface shape, and the film of the reservoir film 250 can be deformed according to the residual amount of the liquid stored in the storage portion 290. Therefore, the fluid can be squeezed out so as not to be left in the storage portion 290. In addition, it is preferable that the reservoir film 250 at this time be processed to have a curved surface shape along the above-mentioned curved surface shape. Accordingly, even when the amount of fluid in the storage portion 290 is reduced, the reservoir film 250 is deformed along the curved surface, and thus the liquid can be squeezed out so as not to be left.

The reservoir film 250 is configured as a multi-layer film. At this time, the inner layer is preferably made of polypropylene, and as the material of the outer layer, a material having excellent gas barrier properties is preferably selected. In addition, the reservoir film 250 is not limited to this, and for example, may be made of a thermoplastic elastomer or may be a film made by pasting another material to the thermoplastic elastomer.

A cartridge septum 280 is provided on the lower surface side of the cartridge 20 (FIG. 9). The cartridge septum 280 is inserted into a cartridge septum insertion hole 241 provided in the cartridge base presser 240 when the cartridge base 210 and the cartridge base presser 240 are assembled. One surface of the cartridge septum 280 is exposed to opening portions 340a and 360a of a patch base 340 and an adhesive tape 360 (FIGS. 2 and 9), and the other surface thereof communicates with a fluid inlet port 211. The fluid inlet port 211 is open between the reservoir film 250 and the cartridge base 210. Therefore, the liquid to be injected using an injection needle or the like via the cartridge septum 280 is stored in the storage portion 290.

Next, the patch 30 (corresponding to an injection portion) will be described with reference to mainly FIG. 4.

The patch 30 includes a catheter 310, an introduction needle 320, an introduction needle folder 321, an introduction needle septum 322, a port base 330, the patch base 340, the patch septum 350, and the adhesive tape 360.

The patch connection needle 231 is inserted through the patch septum 350 as described later to supply the liquid to the patch 30. The patch septum 350 is provided in the side wall portion of the patch 30, and accordingly, the patch connection needle 231 penetrates through the patch septum 350 when the reservoir 20 is mounted toward the side surface of the patch 30.

In addition, the septum of the patch septum 350 and the like is formed of a material (for example, silicone rubber, isoprene rubber, or butyl rubber) so that a hole that is open due to the penetration of a needle or the like is blocked. Accordingly, even when the needle is inserted through and removed from the septum, the liquid or the like does not leak out via the septum.

The catheter 310 is a tube for injecting the liquid. A portion of the catheter 310 is held by the port base 330, and a portion thereof is exposed on the lower side of the port base 330. In order to inject the liquid by using the patch 30, the exposed portion of the catheter 310 is placed into a living body or the like and the liquid is continuously injected. Therefore, the catheter 310 is formed of a soft material such as a fluororesin or a polyurethane resin having excellent biocompatibility.

The introduction needle 320 is a hollow long and thin needle-like member, and the external shape thereof is smaller than the inside diameter of the catheter 310. The introduction needle 320 is inserted into the catheter 310 before use. The sharp end side of the introduction needle 320 is exposed in the downward direction of the catheter 310, and the other end side thereof is fixed to the introduction needle folder 321. In addition, before use, the introduction needle 320 is inserted through the introduction needle septum 322 fixed into the port base 330.

In this configuration, the introduction needle 320 is drawn from the inside of the catheter 310 by drawing the introduction needle folder 321 from the port base 330. However, the liquid that flows from the patch connection needle 231 does not leak out from the introduction needle septum 322 side and flows into the living body through the catheter 310.

The patch 30 includes the patch base 340. The patch base 340 is fixed to the port base 330, includes a cartridge fixing member 341, and has a function of fixing the cartridge 20 to the patch 30. In order to connect the cartridge 20 to the patch 30, the cartridge 20 is moved to slide from the left of FIG. 2 toward the patch 30. In addition, the patch connection needle 231 provided in the cartridge 20 penetrates through the patch septum 350 and is inserted into the patch 30.

The patch base 340 includes the adhesive tape 360 at the lower surface thereof. In addition, the micropump 1 is able to stick to the living body or the like.

In the above configuration, when the main body 10 and the cartridge 20 are assembled in one body, the clogging detection element 123 is disposed above the clogging detection window 223, and the bubble detection element 124 is disposed above the bubble detection window 224. Accordingly, by monitoring the tube 225, occurrence of clogging of the liquid and occurrence of bubbles in the tube 225 can be detected.

In addition, when the main body 10 and the cartridge 20 are assembled, the cam 121 of the main body 10 is inserted into a cam accommodation portion 227c of the finger base 227. Accordingly, the cam surface 121a of the cam 121 is disposed at a position that opposes the rear end portion 222b of the finger 222. In addition, as the cam 121 is rotated, the cam surface 121a abuts on the rear end portion 222b of the finger 222 to enable the finger 222 to slide.

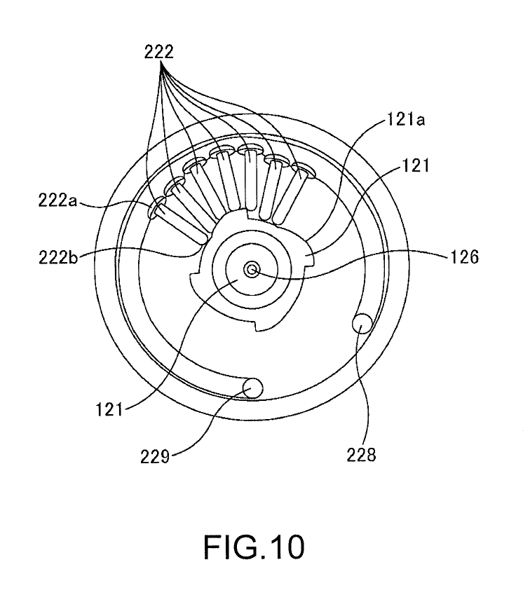

FIG. 10 is a diagram illustrating a rotary finger pump. The cam 121 is provided with four cam noses. Each of the cam noses has a shape in which the height thereof is transited to be gradually increased from the lowest portion of the cam nose to the highest portion, and when the height reaches the highest portion, the height is transited to the lowest portion of the adjacent cam nose. By employing this shape, when the cam 121 is rotated, the tip end portions 222a of the plurality of fingers 222 sequentially press the tube 225 in a direction from the suction connector 228 side to the discharge connector 229 side. In addition, the liquid in the tube 225 can be sent to the discharge connector 229 side from the suction connector 228 side.

In this configuration, the tube 225, the finger unit 220, the cam 121, and the piezoelectric motor 150, which correspond to a pump portion, are disposed closer to the outer side than the storage portion 290 with respect to the living body, and thus the storage portion 290 that stores the liquid can be protected by the pump portion. In addition, the storage portion 290 is less likely to be broken.

Further, a reduction in the size of the micropump 1 provided with the storage portion 290 and the pump portion is preferable. Through the laminated arrangement described above, a further reduction in the size can be realized. At this time, since the storage portion 290 is provided on the living body side, the temperature of the liquid in the storage portion 290 can be kept by the body temperature of the living body.

### Other Embodiments

The micropump 1 described above achieves a reduction in size and a reduction in thickness and thus allows a small amount of fluid to stably and continuously flow. Therefore, the micropump 1 is mounted into a living body or on the surface of a living body and is appropriate for medical uses such as the development of new drugs or drug delivery. In addition, in various mechanical devices, the micropump 1 may be mounted inside the device or outside the device to be used for transport of fluid such as water, a saline solution, liquid medicine, oils, aromatic liquid, ink, or gas. Moreover, the micropump as a single member can be used for flowing or supplying a fluid.

The above-described embodiments are for facilitating the understanding of the invention and should not be construed to limit the invention. The invention can be modified and improved without departing from the spirit and naturally includes the equivalents thereof.

The entire disclosure of Japanese Patent Application No. 2012-228262, filed Oct. 15, 2012 is expressly incorporated by reference herein.

What is claimed is:

1. A fluid injecting apparatus comprising:
a storage portion which stores a fluid; and
a pump portion which transports the fluid,
wherein the storage portion has a housing and a film, and
the housing is disposed between the pump portion and the film.

2. The fluid injecting apparatus according to claim 1,
wherein the pump portion includes a tube in which the fluid flows, a plurality of fingers which press the tube, a cam which moves the fingers, and a motor which drives the cam to rotate.

3. The fluid injecting apparatus according to claim 2, further comprising:
a main body portion; and
a cartridge portion,
wherein the tube, the plurality of fingers, and the storage portion are stored in the cartridge portion,
the motor and the cam are stored in the main body portion, and
the cartridge portion and the main body portion are able to be locked to each other, and the cam is able to be engaged with the fingers.

4. The fluid injecting apparatus according to claim 3,
wherein the housing is made of a plastic, and
the film is made of a film.

5. The fluid injecting apparatus according to claim 4,
wherein an upper portion of the housing made of the plastic is configured as a curved surface.

6. The fluid injecting apparatus according to claim 4,
wherein the cartridge portion includes a first septum which transports the fluid.

7. The fluid injecting apparatus according to claim 3, further comprising:
a transporting portion which transports the fluid of the cartridge portion,
wherein the transporting portion includes a second septum that transports the fluid from the cartridge portion, and a catheter that transports the fluid transported via the second septum.

8. The fluid injecting apparatus according to claim 7,
wherein the cartridge portion includes a needle member that is inserted through the second septum to send the fluid to the transporting portion, and
a tip end position of the needle member has the same height as that of the storage portion in a height direction in which the pump portion and the storage portion overlap.
